(19) United States
(12) Patent Application Publication
(10) Pub. No.: US 2015/0313823 A1
(43) Pub. Date: Nov. 5, 2015

(54) CATIONIC PYRITHIONE DISPERSION

(71) Applicant: ARCH PERSONAL CARE PRODUCTS, LP, South Plainfield, NJ (US)

(72) Inventors: Falen D. LOCKETT, Mableton, GA (US); Diana CICCOGNANI, Cumming, GA (US)

(21) Appl. No.: 14/651,025

(22) PCT Filed: Dec. 20, 2013
(86) PCT No.: PCT/US13/77201

§ 371 Date: Jun. 10, 2015

Related U.S. Application Data
(60) Provisional application No. 61/740,551, filed on Dec. 21, 2012.

(51) Int. Cl. A61K 8/49; A61Q 5/12; A61Q 5/02; A61K 8/41; A61K 8/42

(52) U.S. Cl. CPC A61K 8/49; A61K 8/416; A61K 8/42; A61Q 5/02; A61Q 5/12; A61K 2800/40

(57) ABSTRACT

The present invention provides a cationic dispersion containing pyrithione compound, water and a cationic surfactant. The cationic surfactant is present in an amount sufficient to disperse the pyrithione in water. The dispersion is useable as an additive to personal care compositions, such as conditioners.

### FIELD OF THE INVENTION

The present invention relates to a cationic dispersion of an active ingredient and, more particularly, a cationic dispersion of a pyrithione compound.

### BACKGROUND OF THE INVENTION

Zinc pyrithione is an antimicrobial active used in many different applications and is very well known as an anti-dandruff active. Products for this application area are primarily shampoos which are anionic surfactant systems; however, as consumers become more sophisticated and look to additional product offerings, formulators are increasingly developing alternative anti-dandruff products with very different characteristics. One example is hair conditioners, which are meant to complement the shampoo products.

While non-cationic zinc pyrithione (ZPT) dispersions have been used in some cationic products, there are some limitations when formulating the non-cationic surfactant stabilized zinc pyrithione. For example, when the anionic formulated zinc pyrithione is mixed into these cationic hair conditioners, the addition of the zinc pyrithione dispersion can destabilize the cationic silicones, due to incompatibility. This is a problem for a number of reasons. For example, one problem is anionic zinc pyrithione dispersions prevent the production of a homogenous product, since the zinc pyrithione flocculates with the silicones. This flocculation then results in a sticky gum which can deposit on the surface of the manufacturing vessels, presenting a big challenge for cleansing and hygiene conditions in the plant.

This incompatibility also limits the concentration of active that can be loaded into the product. In an anionic shampoo, it is common to have about 1% active in the formulation. But in conditioner, lower concentrations are used and this is probably because the formulation would be unattractive if higher levels of active were formulated into the conditioner. Further, it has been demonstrated that the more zinc pyrithione deposited on the scalp, the better the control of dandruff. So it follows that higher dosages of zinc pyrithione in the product would give a more efficacious product.

The anionic surfactant used in most zinc pyrithione dispersions is sodium polynaphthalene sulfonate. This surfactant disperses the zinc pyrithione by imparting a negative charge around the particles so that they are electrostatically repelled from each other. This aids in preventing the zinc pyrithione particle from packing together when the particles settle. Sodium polynaphthalene sulfonate also has the unique quality that small quantities of it will liquefy a zinc pyrithione cake (zinc pyrithione and water), thereby making the zinc pyrithione easier to handle. It is these qualities that have resulted in its widespread use in anionic types of dispersions. But these qualities come with a down side which is addressed here. A study was undertaken to investigate how to make alternative dispersions with different additives that would be suited to these problematic cationic formulations.

It has been discovered that zinc pyrithione may be dispersed in a cationic dispersion and this cationic dispersion avoids the problems caused by the addition of an anionic dispersion into cationic composition. The present invention provides a way to place a pyrithione active ingredient into a cationic composition, such as a hair conditioner or a conditioning shampoo.

### SUMMARY OF THE INVENTION

In one aspect, the present invention provides a cationic dispersion containing pyrithione compound, water and a cationic surfactant. The cationic surfactant is present in an amount sufficient to disperse the pyrithione in water.

The dispersion of the present invention is useable as an additive to personal care compositions, such as conditioners and conditioning shampoos.

These and other aspects will become apparent when reading the detailed description of the invention.

### DETAILED DESCRIPTION OF THE INVENTION

It has now been surprisingly found that pyrithione compounds, such as zinc pyrithione, may be cationic dispersed into water using a cationic surfactant and that this cationic dispersion is easily added to cationic personal care products or other cationic systems.

Pyrithione compounds which may be used include metal pyrithione. Examples of metal pyrithione compound include metal pyrithione compounds such as zinc pyrithione, copper pyrithione, zirconium pyrithione, bismuth pyrithione, and the like. When it comes to personal care compositions, zinc pyrithione is known to be cost effective and is a very effective agent to treat dandruff.

Generally, the water in the dispersion may be distilled water, deionized water or tap water. It is generally preferred that water is softer water rather than water containing minerals, such as calcium and magnesium, which tends to make the water hard. However, any water may be used to prepare the dispersion of the present invention.

For the cationic surfactant, any cationic surfactant that is compatible with the pyrithione compound may be used. However, when the dispersion is to be used in a personal care product, such as a cationic conditioner, it is generally preferred that cationic surfactants that are acceptable or approved for these personal care compositions be used.

The most common principle active ingredients in hair conditioners are quaternary ammonium compounds, which are referred to in the art as “quats”. Quaternary means that the ammonium nitrogen atom which has 4 chemical groups attached. Quats can be thought of as ammonium salts with hydrogen molecules replaced by alkyl groups. The anion is usually chloride. A typical quat, for example, is cetyl trimethyl ammonium chloride. A quat's ability to condition comes from the hydrophobic (water-repelling) nature of the long C—H-tail and the positive (cationic) charge of the N-group which is attracted to the negatively (anionic) charged proteins of the hair. This electrostatic interaction, coupled with the fatty nature of long C—H-tail, inhibits rinse-off and makes the hair cuticle smooth, soft and lubricious. As a result, the hair can be combed much more easily and fly-away is largely reduced due to the neutralization of the static electrical build up.

The fatty chains (C—H-tails) are derived from various oily components as coconut oil, castor oil, canola oil or others. The longer the fatty chain the more lubricious is the quat. Hence, for dry and damaged hair, quats with long or multiple fatty chains are preferred (e.g., behentrimonium or propyltrimonium). Exemplary cationic surfactants used in conditioners include the following: behentrimonium chloride, behentrimonium methosulfate, benzalkonium chloride, cetrimonium chloride, cinnamidopropyltrimonium chloride, cocotrimonium chloride, dicetyldimonium chloride, dicocodimonium chloride, dihydrogenated tallow dimethylammonium chloride, hydrogenated palm trimethylammonium chloride, laurtrimonium chloride, quaternium-15, quaternium-18 bentonite, quaternium-18 hectonite, quaternium-22, stearalkonium chloride, tallowtrimonium chloride, tricetyldimonium chloride, cetrimonium bromide, and alkyl amines, such as stearamidopropyl dimethylamine (lactate, citrate, propionate), isostearamidopropyl dimethylamine, isostearamidopropyl morpholine, wheatgermamidopropyl dimethylamine, and behenamidopropyl dimethylamine. It is believed that any of these cationic surfactants may be used in the present invention. It is further noted that some quats, such as benzalkonium chloride and cetrimonium chloride, may also impart further antimicrobial properties to the product which the cationic dispersion is added. Cetrimonium chloride is used as one of the surfactants in the examples that follow.

The following cationic surfactants listed in Table 1 have been tested and have been shown to be effective at dispersing the metal pyrithione compound in water.

TABLE 1

| Suitable cationic | | | |
|---|---|---|---|
| surfactants | | CAS identifier | Structure |
| #1 | Cetethyl Morpholinium Ethosulfate | CAS# 78-21-7 | 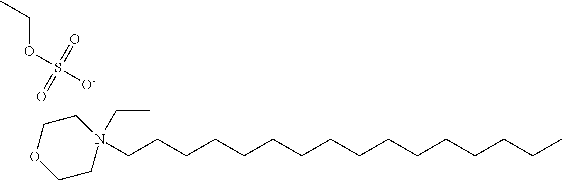 |
| #2 | Bishydroxyethyl | CAS# 70025-19-3 | — |
| | Dihydroxypropyl | | |
| | Stearamonium | | |
| | Chloride | | |
| #3 | Cetrimonium Chloride | CAS# 112-02-7 | 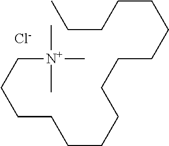 [SMILES: CCCCCCCCCCCCCCCC[N+](C)(C)C ; [Cl-]] |
| #4 | Olealkonium Chloride | CAS# 37139-99-4 | 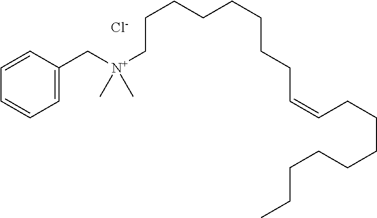 [SMILES: CCCCCCCC/C=C\CCCCCCCC[N+](C)(C)Cc1ccccc1 ; [Cl-]] |
| #5 | Dicetyldimonium Chloride | CAS# 1812-53-9 | 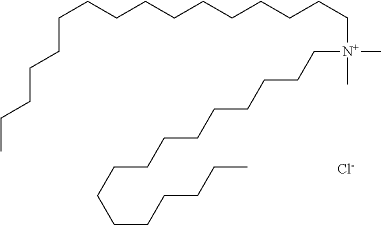 [SMILES: CCCCCCCCCCCCCCCC[N+](C)(C)CCCCCCCCCCCCCCCC ; [Cl-]] |

The dispersion of the present invention is generally rich in the pyrithione compound. This is because the pyrithione dispersion is intended to be added to a composition so that the amount of the pyrithione compound is within a desired range of the formulator. As such, the dispersion is generally added as a portion to the other ingredients in the personal care composition. For example, a dispersion containing 25 wt. % of the pyrithione compound will be added in 1 part per 100 parts, on a weight basis, of the personal care composition to obtain a personal care composition containing about 0.25 wt. % pyrithione. Therefore, it is generally desired that the dispersion contain as much pyrithione as possible, but the dispersion should still be compatible and mixable with the cationic personal care product.

Generally, the dispersion of the present invention will contain between about 1.0 to about 50 percent by weight of the pyrithione compound. Typically, the pyrithione compound is present in an amount between about 5.0 and 40 percent by weight of the dispersion and more typically between about 10 and 35 percent by weight of the dispersion. The amount of the pyrithione compound is generally limited to the ability of the selected surfactant to disperse the pyrithione compound in the water.

The dispersion of the present invention will be generally up to about 50 percent by weight pyrithione compound, up to about 20 percent by weight of the cationic surfactant and the remainder water. The amount of the pyrithione compound is generally limited by the ability to disperse the pyrithione compound in water. Generally, a mixture of about 50 percent by weight of zinc pyrithione and the remainder water is a thick paste or cake-like structure. Therefore, the ability to disperse the pyrithione in water is limited by the amount of water available to disperse the pyrithione in the water.

With respect to the amount of surfactant, as a set weight percentage of the pyrithione compound, the surfactant must replace water. Higher surfactant levels can be beneficial for the composition the dispersion is being added. For example, the cationic surfactants in the personal care composition may be introduced into the personal care composition via the dispersion. Generally, the surfactant will be present in an amount up to about 20 percent by weight of the dispersion. Typically, the cationic surfactant will be present in an amount about 0.1 to about 10 percent by weight, more typically, between about 0.25 and about 5 percent by weight and most typically between about 0.5 and about 2.5 percent by weight. Exemplified amounts of surfactant are about 0.625, 1.25 and 2.5 percent by weight of the surfactant.

The dispersion will also contain the balance of the composition as water. Generally, the water will be present in an amount of at least about 40 percent by weight of the dispersion. Below about 40 percent by weight water, there will not be enough water to disperse the pyrithione compound. Typically, the dispersion will contain about at least 50 percent by weight water and more typically at least 60 percent by weight water. The upper limit of the amount of water is generally about 99 percent by weight.

Other additives may be present in the dispersion, so long as the additional ingredients do not disrupt the stability of the dispersion. For example, salt may be present in an amount up to about 4 percent by weight. Other similar ingredients may be present, such as, for example antifoaming agents, thickeners, preservatives and the likes may be added to the dispersion. Preservatives or biocides are added to prevent bacterial contamination and the like from adversely affecting the dispersion. Exemplary preservative include, isothiazolinone compounds, such as benzisothiazolinone, methyl isothizolinone, 5-Chloro-2-Methyl-4-Isothiazolin-3-One OMIT and mixtures thereof.

To form the dispersion, the ingredients of the dispersion are mixed and subjected to mixing using mixing methods. Suitable mixing methods include, but are not limited to, high-shear mixing and/or sonication. Exemplary high-shear mixing may be accomplished using a stir bar or an overhead mixer. The mixing occurs for a period of time sufficient to form a dispersion or suspension of the pyrithione compound ingredient in the water. The period of time may vary depending on the mixing conditions used. For example, the mixing accomplished by sonification may take less time than mixing accomplished using high shear mixing. The result of the mixing is a dispersion of the pyrithione compound in water with a cationic surfactant.

The advantages provided by the present invention providing a stable cationic pyrithione compound, in particular zinc pyrithione, dispersion product that is suitable for formulating into a wide variety cationic and non-ionic formulations, as well as anionic formulations, such as conditioning shampoos. This cationic dispersion may be formulated with commonly accepted personal care raw materials, making it especially suitable for formulating into cationic and non-ionic personal care formulations, such as hair conditioners, as well as anionic formulations, such as conditioning shampoos. This will provide a conditioner that will have an antidandruff effect. In addition, the stable cationic pyrithione compound dispersion product will allow for a higher active content to be homogenously formulated into conditioners type products. Further, the cationic dispersion may also reduce the need for additional ingredients, such as thickeners to help keep the pyrithione compound suspended in the dispersion. Last, but not least, the dispersion of the present invention will also provide a stable cationic zinc pyrithione dispersion product that makes it easier to formulate the final product since sticky gums are not formed by the addition of a cationically dispersed zinc pyrithione into the conditioners type products.

The present invention is further described in detail by means of the following Examples. All parts and percentages are by weight and all temperatures are degrees Celsius unless explicitly stated otherwise.

### EXAMPLES

### Preparation of Cationic Dispersions

### Example 1

Zinc pyrithione wet cake made up at 48 wt. % zinc pyrithione and 52 wt. % water was weighed out. To this wet cake 1.25 wt. % by weight surfactants were added and the resulting composition was mixed for a period of time using a high-speed overhead mixer. For comparison purposes, an example containing no surfactant, and example containing the anionic surfactant sodium polynaphthalene sulfonate and an example within the scope of the present invention containing the cationic surfactant bis-hydroxyethyl-dihydroxypropyl stearammonium chloride (surfactant #2 in Table 1) were formed. The comparative example C2 contains 0.25% by weight of carboxymethyl cellulose, which is conventionally added to anionic dispersions as a thickener. In addition, the resulting composition was attempted to be added to commercially available cationic conditioner. The commercially available conditioners used for this test are as follows: Clear Scalp & Hair Therapy Nourishing Daily Conditioner, Total Care which contains: Water (Aqua), Cetearyl Alcohol, Cyclopentasiloxane, Stearamidopropyl Dimethylamine, Dimethiconol, Fragrance, Behentrimonium Chloride, Dipropylene Glycol, Lactic Acid, Zinc Pyrithione, DMDM Hydantoin, Zinc Sulfate, Sodium Chloride, TEA Dodecylbenzenesulfonate, PEG 150 Distearate, Tocopheryl Acetate, Prunus Amygdalus Dulcis (Sweet Almond) Oil, Panthenol, Lysine HCl, Hydrogenated Coconut Oil, Helianthus Annuus (Sunflower) Seed Oil, Glycine Soja (Soybean) Oil, Glycerin, Methylisothiazolinone, Niacinamide, Aloe Barbadensis Leaf Extract, Ascorbic Acid, Methylchloroisothiazolinone, Red 33 (CI 17200);

TRESemme Naturals Nourishing Moisture Conditioner, Aloe Vera and Avocado which contains: Water, Stearyl Alcohol, Cetyl Alcohol, Capric/Caprylic Triglyceride, Stearamidopropyl Dimethylamine, Fragrance, Lauryl Lysine, Caprylyl Glycol, Brassica Campestris (Rapeseed)/Aleurites Fordi Oil Copolymer, Aspartic Acid, Distearyldimonium Chloride, Persea Gratissima (Avacado) Oil (Organic), Citric Acid, Alcohol, Isopropyl Alcohol, Aloe Vera (Aloe Barbadensis) Leaf Juice (Organic); and

Suave Naturals Conditioner which contains: Water (Aqua), Cetyl Alcohol, Stearamidopropyl Dimethylamine, Fragrance (Parfum), Lactic Acid, Potassium Chloride, Disodium EDTA, DMDM Hydantoin, PEG-150 Distearate, Tocopheryl Acetate (Vitamin E Acetate), Methylchloroisothiazolinone, Methylisothiazolinone, Spirulina Maxima Extract, Mentha Aquatica Leaf Extract, Nymphaea Alba Flower Extract, Blue 1 (CI 42090), Red 33 (CI17200)

The results are shown in Table 2.

TABLE 2
Dispersions with 48 wt. % active ZPT

| EXPT | Surfactant | Active | Surfactant | CMC | | Evenly disperses |
|---|---|---|---|---|---|---|
| # | # | ZPT % | % | % | Appearance | in conditioner? |
| C1 | No surfactant | 48% | 0.00% | 0% | Very viscous, paste | N/A |
| | | | | | like material | |
| C2 | Control | 48% | 1.25% | 0.25% | Liquid, visible | No |
| | anionic dispersion | | | | separation and | |
| | | | | | settling (minor) | |
| 1 | #2 | 48% | 1.25% | 0% | Liquid, very | Yes |
| | | | | | little separation | |

The results show cationic surfactant dispersion was easily added to a commercially available hair conditioner and will evenly disperse into the conditioner.

### Example 2

Example 1 was repeated, however, the final dispersions contained 25 wt. % zinc pyrithione. Additional surfactants shown in Table 1 were tested and the surfactants correspond to the surfactants as numbered in TABLE 1. It was found with the additional water present, it was easier to form dispersions which are more fluid like, which will aid in the addition of the dispersion to personal care compositions, such as conditioners. As with Example 1, the components were mixed using an overhead mixer. In this example, some of the cationic surfactant containing compositions were also tested with carboxymethyl cellulose (CMC) as is shown in Table 3.

All dispersions were allowed to stand and then tested for degree of settling and ease of re-suspension.

Table 3 outlines the characteristics of the formulations put together being the 25% active dispersions.

TABLE 3
Dispersions with 25% active ZPT

| | | | | | | Homogeneously | |
|---|---|---|---|---|---|---|---|
| | Surfactant | Active | Surfactant | CMC | | disperses in | |
| ex | # | ZPT % | % | % | Appearance | conditioner? | Notes |
| C3 | No | 25% | 0.00% | 0% | Viscous, paste like | Yes | — |
| | surfactant | | | | material | | |
| C4 | Control | 25% | 1.25% | 0% | Liquid, visible | No | — |
| | anionic | | | | separation and | | |
| | | | | | major settling | | |
| C5 | Control | 25% | 1.25% | 0.25% | Liquid, visible | No | — |
| | anionic | | | | separation and | | |
| | | | | | minor settling | | |
| 2 | #1 | 25% | 1.25% | 0% | Liquid, visible | Yes | fairly easy to re |
| | | | | | separation and | | mix settled |
| | | | | | minor settling | | material |
| 3 | #1 | 25% | 1.25% | 0.25% | Liquid, visible | Yes | fairly easy to re |
| | | | | | separation and | | mix settled |
| | | | | | minor settling | | material |
| 4 | #2 | 25% | 1.25% | 0.00% | Liquid, visible | Yes | moderately easy |
| | | | | | separation and | | to re mix settled |
| | | | | | major settling | | material |
| 5 | #2 | 25% | 1.25% | 0.25% | Liquid, visible | Yes | much less |
| | | | | | separation and | | separation than |
| | | | | | very minor settling | | sample without |
| | | | | | | | CMC |
| 6 | #3 | 25% | 1.25% | 0.00% | Liquid, visible | Yes | fairly easy to re |
| | | | | | separation and | | mix settled |
| | | | | | major settling | | material |
| 7 | #3 | 25% | 1.25% | 0.25% | Liquid, visible | Yes | fairly easy to re |
| | | | | | separation and | | mix settled |
| | | | | | minor settling | | material |
| 8 | #4 | 25% | 1.25% | 0.00% | Liquid, visible | Yes | fairly easy to re |
| | | | | | separation and | | mix settled |
| | | | | | major settling | | material |
| 9 | #4 | 25% | 1.25% | 0.25% | Liquid, visible | Yes | much less |
| | | | | | separation | | separation than |
| | | | | | | | sample without |
| | | | | | | | CMC |
| 10 | #5 | 25% | 1.25% | 0.00% | Viscous liquid, very | Yes | |
| | | | | | little separation | | |

It is possible that with further formulation work, higher concentrations might be achievable with a greater range of surfactants but given that conditioners usually have lower concentrations of zinc pyrithione, a 25 percent by weight active level is an acceptable concentration for this type of product.

While the invention has been described above with references to specific embodiments thereof, it is apparent that many changes, modifications and variations can be made without departing from the invention concept disclosed herein. Accordingly, it is intended to embrace all such changes, modifications, and variations that fall within the spirit and broad scope of the appended claims.

1. A cationic dispersion comprising a metal pyrithione compound, water and a cationic surfactant, wherein the cationic surfactant is present in an amount sufficient to disperse the metal pyrithione compound in the water, the metal pyrithione compound is present between 1 and 50% by weight of the dispersion, the surfactant is present in an amount between 0.1 and 20% by weight and the balance is water.

2. The cationic dispersion according to claim 1, wherein the metal pyrithione compound comprises zinc pyrithione.

3. The cationic dispersion according to claim 1, wherein the metal pyrithione compound is present in an amount between 5.0 and 40% by weight of the dispersion, the surfactant is present in an amount between 0.1 and 10% by weight and the balance is water.

4. The cationic dispersion according to claim 3, wherein the metal pyrithione compound is present in an amount between 10 and 35% by weight of the dispersion, the surfactant is present in an amount between 0.25 and 5% by weight and the balance is water.

5. The cationic dispersion according to claim 4, wherein the metal pyrithione compound is present in an amount between 10 and 35% by weight of the dispersion, the surfactant is present in an amount between 0.5 and 2.5% by weight and the balance is water.

6. The cationic dispersion according to claim 1 wherein the cationic surfactant is selected from the group consisting of cetethyl morpholinium ethosulfate, bishydroxyethyl dihydroxypropyl stearamonium chloride, cetrimonium chloride, olealkonium chloride, dicetyldimonium chloride, benzalkonium chloride and mixtures thereof.

7. The cationic dispersion according to claim 6, wherein the metal pyrithione compound comprises zinc pyrithione.

8. The cationic dispersion according to claim 5 wherein the cationic surfactant is selected from the group consisting of cetethyl morpholinium ethosulfate, bishydroxyethyl dihydroxypropyl stearamonium chloride, cetrimonium chloride, olealkonium chloride, dicetyldimonium chloride, benzalkonium chloride and mixtures thereof.

9. The cationic dispersion according to claim 8, wherein the metal pyrithione compound comprises zinc pyrithione.

10. The cationic dispersion according to claim 9, wherein the metal pyrithione compound is present in an amount between 5.0 and 40% by weight of the dispersion, the surfactant is present in an amount between 0.1 and 10% by weight and the balance is water.

11. The cationic dispersion according to claim 10, wherein the metal pyrithione compound is present in an amount between 10 and 35% by weight of the dispersion, the surfactant is present in an amount between 0.25 and 5% by weight and the balance is water.

12. The cationic dispersion according to claim 11, wherein the metal pyrithione compound is present in an amount between 10 and 35% by weight of the dispersion, the surfactant is present in an amount between 0.5 and 2.5% by weight and the balance is water.

13. A personal care composition comprising a cationic dispersion according to claim 1.

14. A personal care composition comprising a cationic dispersion according to claim 10.

15. The personal care composition according to claim 13, wherein the composition is a hair conditioner.

16. The personal care composition according to claim 13, wherein the composition is a conditioning shampoo.

17. The personal care composition according to claim 14, wherein the composition is a hair conditioner.

18. The personal care composition according to claim 14, wherein the composition is a conditioning shampoo.
